(19) United States
(12) Patent Application Publication
(10) Pub. No.: US 2015/0072302 A1
(43) Pub. Date: Mar. 12, 2015

(54) LIGHT ACTIVATED TOOTH WHITENING COMPOSITION

(71) Applicant: JASIBO, LLC., Las Vegas, NV (US)

(72) Inventor: Jennifer JABLOW, New York, NY (US)

(73) Assignee: JASIBO, LLC., Las Vegas, NV (US)

(21) Appl. No.: 14/256,157

(22) Filed: Apr. 18, 2014

Related U.S. Application Data
(60) Provisional application No. 61/814,183, filed on Apr. 19, 2013.

(51) Int. Cl. A61C 19/06; A61K 8/22; A61Q 11/00; A61K 8/27

(52) U.S. Cl. CPC A61C 19/066; A61K 8/27; A61K 8/22; A61Q 11/00; A61K 2800/10
USPC 433/29; 433/215; 424/53

(57) ABSTRACT

A dental bleaching agent and a dental bleaching method by which the shortcomings associated with the conventional techniques are overcome, and a better teeth whitening result is accomplished.

### CROSS-REFERENCE TO RELATED APPLICAITONS

This application claims priority to and the benefit of U.S. Provisional Patent Application Ser. No. 61/814,183, filed on 19 Apr. 2013, the disclosure of which is incorporated herein by reference in its entirety.

### FIELD

The aspects of the present disclosure generally relate to teeth whitening products for home use, and in particular, a method for bleaching discoloration from the teeth using an LED light source, a pulsating light (or not pulsating), hydrogen peroxide and a zinc oxide to accelerate the breakdown of hydrogen peroxide.

### BACKGROUND

Teeth whitening in the dental office, and at home has become a billion dollar industry. Professional dental procedures will generally include an in-office application of a high concentration peroxide paint or film followed by light activated whitening. Such high concentration peroxide products are not suitable for home use.

Take-home trays or strips and currently available commercial at-home products are characterized by substantially lower peroxide concentration. The home use methods including strips and paint-on products, frequently include at-home light sources that claim to work by light activation of the peroxide.

Heat is also additionally employed as an adjunct peroxide activator. Unfortunately, heat causes undesirable dehydration and sensitivity of teeth. Thus, there is a need to develop dental products for home use with safe concentrations of peroxide that can provide desired whitening results in a reasonable period.

Accordingly, it would be desirable to provide a teeth whitening product for home use that resolves at least some of the problems identified above.

### SUMMARY

As described herein, the exemplary embodiments overcome one or more of the above or other disadvantages known in the art.

One aspect of the present disclosure is directed to a dental bleaching agent and a dental bleaching method by which the shortcomings associated with the conventional techniques are overcome, and a better teeth whitening result is accomplished.

More specifically, the aspects of the present disclosure are directed to a novel method for accelerating the hydrogen peroxide bleaching or whitening reaction against dental stains or discolorations. More specifically, hydrogen peroxide combined with zinc oxide in the presence of concentrated light results in a formulation with enhanced or synergistic dental bleaching properties. Coupling zinc oxide on the tooth surface at the same time as hydrogen peroxide enhances the photo catalytic activity, and the desirable effect of teeth whitening occurs. The methods described above may be single application or may be multiple applications such as 3 sequential applications.

Suitable light illumination, including sources such as Xenon, Quartz, Halogen, Tungsten, and (light emitting diode) LED, for example, can be utilizied. Preferably the light source is an LED light source set to pulsate at a certain frequency, or at a steady stream of light without pulsation.

Another aspect of the present disclosure relates to a method of tooth whitening, wherein the tooth surface is irradiated with a LED light source which accelerates the bleaching activity of the tooth. More specifically, the light source, such as an LED light source, is a low or no heat light source.

Another aspect of the present disclosure relates to a method of whitening wherein the tooth surface is irradiated with a pulsating or non-pulsating LED light source which accelerates the bleaching activity of the tooth. Another aspect of the present disclosure relates to a method of tooth or teeth whitening, wherein the wavelength of light being utilized in the teeth whitening process is in a range from approximately 410 nm to 520 nm, including 460 nm to 520 nm, in the visible blue light spectrum.

Another aspect of the present disclosure relates to a method of whitening teeth, wherein the light being utilized during the teeth whitening process pulsates at a frequency between approximately 1 to 100 HZ. The light sources generally described herein may also be battery operated or with direct or alternating current. Rechargeable battery systems have particular interest.

In one aspect, the illumination device of the present disclosure is suitably shaped to follow the natural curvature of the upper and/or lower arch of the teeth enabling even distribution and disbursement of light to the teeth (preferably anterior). Such illumination devices may also include a handle, a body and a head.

Cooling mechanisms are also of particular interest because of the desire to reduce the temperature in the oral cavity during the bleaching process. Likewise blue light filters and polarimeters enhance the photo catalytic effect of the light source.

Certain light/illumination devices are described in U.S. Provisional Application 61/873,088 filed Sep. 3, 2013, the disclosure of which is hereby incorporated by reference in its entirety.

Another aspect of the present disclosure relates to a method for whitening teeth comprising: applying a zinc oxide to a tooth surface; adding a hydrogen peroxide to the zinc oxide and illuminating the resulting admixture and tooth surface with a visible spectral light source.

Another aspect of the present disclosure relates to a method of whitening teeth wherein said zinc oxide is Zinc Oxide USP formulated as a gel, resin, dentifrice, paste, thin film, strip, powder, liquid, spray, wax or paint (preferably a gel) and applied to the tooth labial surface.

Another aspect of the present disclosure relates to a method of whitening teeth, wherein the zinc oxide is a powder USP, crystal, nanoparticle, spray dry nanomatrix particle dissolved or suspended or in a gel or neat formulation at a concentration of between 0.1 to 30 percent by weight. Zinc oxide formulations of 20 percent, more preferably 10 percent, active are suitable formulations particularly when the opacity of the formulation is low or semi-clear.

Wax formulations are another specific embodiment of the present disclosure. Wax includes synthetic and natural waxes such as paraffins, beeswax, ouricury wax, sugarcane wax, retamo wax, lanolin, petroleum jelly, vegetable waxes (including Bayberry wax, candelilla wax, carnauba wax, Castor wax, Esparto wax, Japan wax, Jojoba oil, Ouricury wax, Rice bran wax or Soy wax). Synthetic fatty acid esters such as cetyl palmitate and myricyl palmitate are additional examples of useful waxes. One specific formulation of water, allyl acrylate copolymer and Hydrogen peroxide is of particular interest.

In one embodiment, waxes and the like may be used to form compositions of peroxide or zinc oxide. Illustrative examples of synthetic polymers used include polyurethanes, polyvinyl chlorides, acrylates, including ethyl vinyl acrylate, polycarbonate, polyphenylene oxide, polyimide, polyethylene, polypropylene, polystyrene, polyvinyl chlorides, polyamides and polyesters, acetates including ethyl vinyl acetate, combinations thereof and art recognized equivalents. Natural materials may also be used, including natural waxes, plant fiber materials such as cellulose, and the like. Preferred materials include beeswax and paraffin. Further, mixtures of such materials may be used.

Another specific composition to be used in conjunction with the aspects of the present disclosure includes paraffin or beeswax. The composition may have a relatively supple consistency such as a gel, foam or wax such that teeth are not damaged by contact therewith. In one embodiment the composition has a gel-like consistency formed from materials including exemplary materials such as gelatin, silicone, gelled mineral oils, and the like. Further, materials such as the Versagel C or M series (Penrenco) or other di-block, tri-block, multi-block and radial block copolymers such as the Kraton G series polymers (Shell Chemical) are suitable for use in the invention. Other exemplary materials that may be used include gelled Permethyl 99A-750, 99A-753-59, and 99A-753-58 tri-block and starburst polymer mixture, OS129880, OS 129881 and OS 129883 from Lubrizol (a styrene/methacrylate copolymer), Viscogel by Laviosa Chimica Mineraria, silicone products such as Medical Adhesive A, 7-9800, RTB 700,732,736 by Dow Corning and dimethicone copolyols such as Dow Corning 3225C and lauryl methicones such as Dow Corning Q2-5200 all by Dow Corning Company.

Another aspect of the present disclosure relates to a method of whitening teeth, wherein the zinc oxide is a powder USP in a gel formulation wherein the average zinc oxide particle size is between 0.01 microns to 100 microns.

Another aspect of the present disclosure relates to a method of whitening teeth, wherein the hydrogen peroxide is a gel, dentrifice, paste, thin film, strip, liquid, spray, or paint applied to the tooth labial surface and creates an admixture with said zinc oxide at the tooth interface.

Another aspect of the present disclosure relates to a method of whitening teeth, wherein the concentration of hydrogen peroxide ranges from 3 to 38 percent, more preferably 3 to 35 percent by weight.

Another aspect of the present disclosure relates to a method of whitening teeth, wherein the admixture of zinc oxide and hydrogen peroxide absorbs most of the irradiated light. Light absorption is affected by numerous factors including opacity of the formulation such as gel, angle of irradiation, surface smoothness or refractions. The formulations of the present disclosure have low to moderate opacity and a dappled tacky texture. The Zinc oxide formulation of the present disclosure may also be adapted to a pH range between about 6 to and including 9. The peroxide formulation of the present disclosure may also be adapted to a pH range between about 4 to and including 7. Upon admixture the modulation of the pH into the alkaline range further enhances the peroxide activity.

Another aspect of the present disclosure relates to a method of whitening teeth, additionally comprising a desensitizing agent such as potassium nitrate, strontium chloride, potassium chloride, potassium citrate, or any mixture thereof.

Another aspect of the present disclosure relates to a method of whitening teeth additionally comprising a light absorbing colorant such as methylene blue or fluorescein. Such agents help by absorbing more of the blue light right at the catalytic interface of the tooth (it makes the activator film slightly yellow in color).

Formulations may also include a remineralizer or a breath freshener.

One formulation of particular interest is a very thin film, with high adherence, good stability in water, fairly opaque, with 5-10 percent zinc oxide concentration.

Another aspect of the present disclosure relates to a teeth whitening kit for home use including: a whitening agent comprising a composition of zinc oxide, a composition of hydrogen peroxide and a light source.

Another aspect of the present disclosure relates to a teeth whitening kit wherein said composition of zinc oxide and said composition of hydrogen peroxide are delivered from a single component with 2 separate chambers that mix upon delivery to the tooth surface. The delivery apparatus for the zinc or peroxide includes one or more of a syringe, ampoule, or tube. Such apparatus may be opaque so as to prevent damage during storage.

The terms “light” or “illumination source” as used herein generally refers to the production of light throughout the visible and UV spectrum at a distance to the teeth, capable of facilitating the reaction of the active agents with the stained teeth. Light sources also may be pulsating at a frequency between about 1 and 100 HZ and a wavelength of about 410 nm to 520 nm, including 460 nm to 520 nm, that is directed at the tooth surface and away from the oral tissues.

Another aspect of the present disclosure relates to the application of the bleaching formulation, wherein the treatment period is from 2-10 minutes, more preferably about 5-7 minutes, more preferably 5 minutes or less. Repeat treatment is also preferred with 2 to 10 sessions being common. More preferably 3-7 sessions (five to seven being most common) are sufficient for achieving desired whiteness. The sessions are typically divided by 24 hours (six to twelve hours also being very common) but can be longer or shorter depending on individual conditions. Total bleaching time is generally between about 20 to 50 minutes, more preferably about 35 minutes total, most preferably about 25 minutes (wherein each session is approximately 5 minutes).

These and other aspects and advantages of the exemplary embodiments will become apparent from the following detailed description considered in conjunction with the accompanying drawings. It is to be understood, however, that the drawings are designed solely for purposes of illustration and not as a definition of the limits of the invention, for which reference should be made to the appended claims. Additional aspects and advantages of the disclosed embodiments will be set forth in the description that follows, and in part will be obvious from the description, or may be learned by practice of the disclosed embodiments. Moreover, the aspects and advantages of the present disclosure may be realized and obtained by means of the instrumentalities and combinations particularly pointed out in the appended claims.

### DETAILED DESCRIPTION OF THE DISCLOSED EMBODIMENTS

The following examples demonstrate methods or uses to successfully whiten a patient's tooth or teeth.

### Example 1

1. Zinc oxide is painted on teeth labial surface in a gel carrier form with the zinc oxide (USP grade) in a concentration of 0.1 to 30% with the size of the particle ranging from 0.01 microns to 100 microns.

2. Hydrogen peroxide gel in a concentration of 3 to 35% is painted on the labial surface of the teeth creating an admixture of the zinc oxide and peroxide at the tooth surface (steps 1 and 2 are interchangeable).

3. A light source pulsating at a frequency between 1 and 100 HZ and a wavelength of 410 nm to 520 nm, alternatively 460 nm to 520 nm, is directed at the tooth surface and away from the oral tissues.

4. After treatment period (e.g., 5, 7, 30 or 35 minutes) the light source is removed. This procedure may be repeated 3-7 consecutive times (such as daily or twice daily) with light source removed after each treatment (e.g., 5, 7, 30 or 35 minutes).

### Example 2

The zinc and peroxide components are maintained in two separate chambers of a pen or syringe and mix at the delivery tip as the components are applied to the tooth. Once the zinc and peroxide components have been applied to the teeth a LED light source is directed at the tooth surface and away from the oral tissues.

Such light source or illumination device is capable of delivering a steady stream of light, preferably pulsating light, at a frequency between about 1 and 100 HZ. Such light source may also provide light throughout the visible and low ultraviolet spectrum but is preferably concentrated in the blue region (410-520 nm), more preferably at a wavelength of 460 to 520 nm.

The illumination device may comprise a mouthpiece and an illuminator device. The illuminator is the source of the light and the mouthpiece may facilitate focusing of the light on the tooth surface (preventing unwanted irradiation of sensitive surfaces such as gum and other soft tissues).

The mouthpiece may comprise dual cylindrical lenses that couple the light from a dual row of LEDs (or other light sources, ideally comprising two rows of 16 light sources tailored to completely and directly illuminate each tooth according to its size, i.e. incisor, cuspid, bicuspid or molar) precisely onto the teeth. Thus, the top and bottom rows of LEDs are focused onto the upper and lower row of teeth respectively. Another embodiment relates to directing the incident light perpendicular to the face of the tooth.

The illuminator may consist of a dual row LED configuration which mates with the dual cylindrical lens array within the mouthpiece. The LEDs may be formed into a curved configuration to mate precisely with the curvature of the lens assemblies on the front surface of the mouthpiece.

The nature of the single axis cylindrical optics is that it also creates high angle irradiance parallel to the direction of the array, which efficiently irradiates the spaces and cracks between the teeth.

Although this technique is suitable for application in dental offices because of the safety and efficacy is importantly useful for home use by the consumer.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit and scope of the invention. Moreover, it is expressly intended that all combinations of those elements and/or method steps, which perform substantially the same function in substantially the same way to achieve the same results, are within the scope of the invention. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

What is claimed is:

1. A method for whitening teeth comprising: applying a zinc oxide to a tooth surface; adding a hydrogen peroxide to the zinc oxide and illuminating the resulting admixture and tooth surface with a visible spectral light source.

2. A method for whitening teeth comprising: applying a hydrogen peroxide to a tooth surface; adding a zinc oxide to the hydrogen peroxide and illuminating the resulting admixture and tooth surface with a visible spectral light source.

3. A method according to claim 1, wherein said zinc oxide is Zinc Oxide USP formulated as a gel and applied to the tooth labial surface.

4. A method according to claim 3, wherein said hydrogen peroxide is a gel applied to the tooth labial surface and forms an admixture with said zinc oxide at the tooth interface.

5. A method according to claim 4, wherein said tooth surface is irradiated with a LED light source which accelerates the bleaching activity of the tooth.

6. A method according to claim 5, wherein said LED light source is a low or no heat light source.

7. A method according to claim 6 wherein said tooth surface is irradiated with a pulsating LED light source which accelerates the bleaching activity of the tooth.

8. A method according to claim 6 wherein said tooth surface is irradiated with a non-pulsating LED light source which accelerates the bleaching activity of the tooth.

9. A method according to claim 3 wherein said zinc oxide is a powder USP in a gel formulation at a concentration of between 0.1 to 30 percent by weight.

10. A method according to claim 9 wherein said zinc oxide is a powder USP in a gel formulation wherein the average zinc oxide particle size is between 0.01 microns to 100 microns.

11. A method according to claim 4, wherein the concentration of hydrogen peroxide ranges from 3 to 35 percent by weight.

12. A method according to claim 6 wherein the wavelength of light is 410 to 520 nm in the visible blue light spectrum.

13. A method according to claim 6 wherein the wavelength of light is 460 to 520 nm in the visible blue light spectrum.

14. A method according to claim 7 wherein the light pulsates at a frequency between 1 to 100 HZ.

15. A method according to claim 11 additionally comprising a desensitizing agent.

16. A method according to claim 15 wherein said desensitizing agent is potassium nitrate, strontium chloride, potassium chloride, potassium citrate, or any mixture thereof.

17. A method according to claim 1 wherein said whitening application is from 2-10 minutes, repeated two to ten sessions.

18. A whitening composition comprising: 0.1 to 30 percent by weight zinc oxide gel and 3 to 35 percent by weight hydrogen peroxide.

19. A whitening composition according to claim 18 wherein each of said zinc oxide and hydrogen peroxide are formulated in a wax.

20. A kit for home use of a whitening agent comprising a composition of zinc oxide, a composition of hydrogen peroxide and a light source.

21. A kit according to claim 20 wherein said composition of zinc oxide and said composition of hydrogen peroxide are delivered from a single component with 2 separate chambers that mix upon delivery to the tooth surface.
